SEQUENCE LISTING (24 sequences)

SEQ ID NO: 1 - protein, 724 aa, Homo sapiens
MPRNSGAGYGCPHGDPSMLDSRETPQESRQDMIVRTTQEKLKTSSLTDRQPLSKESLNHALELSVPEKVNNAQWDAPEEALWTTRADGRVRLRIDPSCPQLPYTVHRMFYEALDKYGDLIALGFKRQDKWEHISYSQYYLLARRAAKGFLKLGLKQAHSVAILGFNSPEWFFSAVGTVFAGGIVTGIYTTSSPEACQYIAYDCCANVIMVDTQKQLEKILKIWKQLPHLKAVVIYKEPPPNKMANVYTMEEFMELGNEVPEEALDAIIDTQQPNQCCVLVYTSGTTGNPKGVMLSQDNITWTARYGSQAGDIRPAEVQQEVVVSYLPLSHIAAQIYDLWTGIQWGAQVCFAEPDALKGSLVNTLREVEPTSHMGVPRVWEKIMERIQEVAAQSGFIRRKMLLWAMSVTLEQNLTCPGSDLKPFTTRLADYLVLAKVRQALGFAKCQKNFYGAAPMMAETQHFFLGLNIRLYAGYGLSETSGPHFMSSPYNYRLYSSGKLVPGCRVKLVNQDAEGIGEICLWGRTIFMGYLNMEDKTCEAIDEEGWLHTGDAGRLDADGFLYITGRLKELIITAGGENVPPVPIEEAVKMELPIISNAMLIGDQRKFLSMLLTLKCTLDPDTSDQTDNLTEQAVEFCQRVGSRATTVSEIIEKKDEAVYQAIEEGIRRVNMNAAARPYHIQKWAILERDFSISGGELGPTMKLKRLTVLEKYKGIIDSFYQEQKM

SEQ ID NO: 2 - DNA, 2172 bp, Homo Sapiens
atgccacgcaattctggagctggatacggctgcccacacggggaccccagcatgctggacagcagagagaccccacaggagagccggcaggacatgattgtgaggaccacccaagaaaaattgaaaaccagctcactgactgacaggcagccactctccaaagagtccctgaaccatgctctcgagctctcagtgccagagaaggtgaataatgcccagtgggatgctccagaggaggcgctgtggacgactcgggccgatgggcgggtgcgcctgcgcatagaccccagctgcccacagcttccctacactgtgcatcggatgttctacgaggccctggataagtatggggacctcatcgctttgggcttcaagcgccaggacaagtgggaacacatctcctactcccaatactacctgctcgcccgcagagccgccaagggcttcctgaagctcggcctgaagcaggcccacagtgtggccatcctcggcttcaactccccggagtggttcttctcggcagtgggcacagtatttgcaggtggcatcgtcactggcatctacaccaccagctccccagaggcctgccagtacatcgcttatgactgctgcgccaatgtcatcatggtcgacacgcagaagcagctggaaaagatcctgaagatctggaaacagttgccacatctaaaggcagtcgtgatatataaagaacctcctccaaacaagatggccaatgtgtacacgatggaggaattcatggagctggggaatgaagtgcctgaggaagccctggacgccatcattgacacccagcagcccaaccagtgctgtgtgctagtctacacttccggcaccactgggaaccccaagggcgtgatgctgagtcaagacaatatcacgtggacggcacggtacggcagccaggccggtgacatccggccggcagaagtccagcaggaggtggtagtcagctacctgcccctcagccatattgccgcccagatctacgacctgtggacaggcatccagtggggggcccaggtttgctttgccgaacccgacgccctgaaggggagcctggtgaacacgctgcgggaggtggagcccacatcacacatgggggtgccccgggtatgggagaagatcatggagcgcatccaggaggtggcggctcagtctggcttcatccggcggaagatgctgctgtgggccatgtcggtgaccttggagcagaacctcacctgccccggcagcgacctgaagcccttcacaaccagactggcagattacctggtgctagccaaggttcgccaggcactgggatttgccaagtgtcaaaagaacttctatggagcggcccccatgatggcagagacacagcacttcttcctgggtctcaacatccgcttgtatgcgggctatggcctcagtgagacctcaggcccccacttcatgtccagtccctacaactaccggctgtacagctcaggcaagttggtgcccggctgtcgggtgaagctggtgaaccaggacgcagagggcattggtgagatctgcctgtggggccgcaccatattcatgggctacctgaacatggaggacaagacttgtgaggccatcgacgaggaaggctggctgcacacgggtgatgctggccgcctggacgccgatggcttcctctacatcactgggcgcctcaaagaattaatcatcacagctggtggggagaatgtgccccctgtgcccatcgaggaggccgtgaagatggagctgcccatcatcagcaatgccatgctcattggggaccagaggaagttcctgtccatgctgctcaccttgaagtgcactctggacccagacacctctgaccagactgataatctgactgaacaagctgtggagttctgccagagggtgggcagcagagccaccacagtgtccgagatcatagagaagaaggatgaggccgtgtaccaggccatcgaagaggggatccggagggtcaacatgaacgcggcggcccggccctaccacatccagaagtgggccattctcgagagagacttctccatttcgggtggagagttgggtcccacgatgaaactgaaacggctcacagttttggagaagtacaaaggtatcattgactccttttaccaagagcaaaaaatg

SEQ ID NO: 3 - protein, 609 aa, Homo Sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 4 - DNA, 1827 bp, Homo Sapiens
atgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtt

SEQ ID NO: 5 - protein, 337 aa, Homo Sapiens
MTNNSGSKAELVVGGKYKLVRKIGSGSFGDVYLGITTTNGEEVAVKLESQKVKHPQLLYESKLYTILQGGVGIPHMHWYGQEKDNNVLVMDLLGPSLEDLFNFCSRRFTMKTVLMLADQMISRIEYVHTKNFLHRDIKPDNFLMGTGRHCNKLFLIDFGLAKKYRDNRTRQHIPYREDKHLIGTVRYASINAHLGIEQSRRDDMESLGYVFMYFNRTSLPWQGLKAMTKKQKYEKISEKKMSTPVEVLCKGFPAEFAMYLNYCRGLRFEEVPDYMYLRQLFRILFRTLNHQYDYTFDWTMLKQKAAQQAASSSGQGQQAQTQTGKQTEKNKNNVKDN

SEQ ID NO: 6 - DNA, 1011 bp, Homo Sapiens
atgacaaacaacagcggctccaaagccgaactcgttgtgggagggaaatacaaactggtgcggaagatcgggtctggctcctttggagacgtttatctgggcatcaccaccaccaacggcgaggaagtagcagtgaagctggaatctcagaaggtcaagcacccccagttgctgtatgagagcaaactctacacgattcttcaaggtggggttggcatcccccacatgcactggtatggtcaggaaaaagacaacaatgtgctagtcatggaccttctgggacccagcctcgaagacctctttaatttctgttcaagaaggttcaccatgaaaactgtacttatgttagccgaccagatgatcagcagaattgaatacgtgcatacaaagaattttctacaccgagacattaaaccagataacttcctgatgggtactgggcgtcactgtaataagttgttccttattgattttggtttggccaaaaagtacagagacaacaggaccaggcaacacataccgtacagagaagataaacacctcattggcactgtccgatatgccagcatcaatgcacatcttggtattgagcagagccgccgagatgacatggaatccttaggctacgttttcatgtattttaatagaaccagcctgccgtggcaaggactaaaggctatgacaaaaaaacaaaaatatgaaaagattagtgagaagaagatgtccacccctgttgaagttttatgtaaggggtttcctgcagaattcgccatgtacttgaactactgtcgtgggctgcgctttgaggaagtcccagattacatgtatctgaggcagctattccgcattcttttcaggaccctgaaccaccaatatgactacacatttgattggacgatgttaaagcagaaagcagcacagcaggcagcctcttccagtgggcagggtcagcaggcccaaacccagacaggcaagcaaactgaaaaaaacaagaataatgtgaaagataac

SEQ ID NO: 7 - protein, 187 aa, Homo Sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 8 - DNA, 561 bp, Homo Sapiens
atggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgat

SEQ ID NO: 9 - protein, 382 aa, Homo Sapiens
MAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAMGIPQAVLPPLPKRPALEKTNGATAVFNTGIFQYQQALANMQLQQHTAFLPPGSILCMTPATSVVPMVHGATPATVSAATTSATSVPFAATATANQIPIISAEHLTSHKYVTQM

SEQ ID NO: 10 - DNA, 1146 bp, Homo Sapiens
atggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccaggctcaatattgtgcatgacacccgctacaagtgttgttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatg

SEQ ID NO: 11 - protein, 393 aa, Homo Sapiens
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACAGRDRRTEEENLRKKGEPHHELPSGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 12 - DNA, 1179 bp, Homo Sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtgctgggagagaccggcgcacagaggaagagaatctccgcaagaaaggggagcctcaccacgagctgccctcagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 13 - protein, 227 aa, Homo Sapiens
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 14 - DNA, 684 bp, Homo Sapiens
atgaacatcaaaggatcgccatggaaagggtccctcctgctgctgctggtgtcaaacctgctcctgtgccagagcgtggcccccttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgcttg

SEQ ID NO: 15 - protein, 440 aa, Homo Sapiens
MGQSQSGGHGPGGGKKDDKDKKKKYEPPVPTRVGKKKKKTKGPDAASKLPLVTPHTQCRLKLLKLERIKDYLLMEEEFIRNQEQMKPLEEKQEEERSKVDDLRGTPMSVGTLEEIIDDNHAIVSTSVGSEHYVSILSFVDKDLLEPGCSVLLNHKVHAVIGVLMDDTDPLVTVMKVEKAPQETYADIGGLDNQIQEIKESVELPLTHPEYYEEMGIKPPKGVILYGPPGTGKTLLAKAVANQTSATFLRVVGSELIQKYLGDGPKLVRELFRVAEEHAPSIVFIDEIDAIGTKRYDSNSGGEREIQRTMLELLNQLDGFDSRGDVKVIMATNRIETLDPALIRPGRIDRKIEFPLPDEKTKKRIFQIHTSRMTLADDVTLDDLIMAKDDLSGADIKAICTEAGLMALRERRMKVTNEDFKKSKENVLYKKQEGTPEGLYL

SEQ ID NO: 16 - DNA, 1320 bp, Homo Sapiens
atgggtcaaagtcagagtggtggtcatggtcctggaggtggcaagaaggatgacaaggacaagaaaaagaaatatgaacctcctgtaccaactagagtggggaaaaagaagaagaaaacaaagggaccagatgctgccagcaaactgccactggtgacacctcacactcagtgccggttaaaattactgaagttagagagaattaaagactatcttctcatggaggaagaattcattagaaatcaggaacaaatgaaaccattagaagaaaagcaagaggaggaaagatcaaaagtggatgatctgagggggaccccgatgtcagtaggaaccttggaagagattattgatgacaatcatgccatcgtgtctacatctgtgggctcagaacactacgtcagcattctttcatttgtagacaaggatctgctggaacctggctgctcggtcctgctcaaccacaaggtgcatgccgtgataggggtgctgatggatgacacggatcccctggtcacagtgatgaaggtagaaaaggccccccaggagacctatgcagatattggggggttggacaaccaaattcaggaaattaaggaatctgtggagcttcctctcacccatcctgaatattatgaagagatgggtataaagcctcctaagggggtcattctctatggtccacctggcacaggtaaaaccttgttagccaaagcagtagcaaaccaaacctcagccactttcttgagagtggttggctctgaacttattcagaagtacctaggtgatgggcccaaactcgtacgggaattgttccgagttgctgaagaacatgcaccgtccatcgtgtttattgatgaaattgacgccattgggacaaaaagatatgactccaattctggtggtgagagagaaattcagcgaacaatgttggaactgctgaaccagttggatggatttgattctaggggagatgtgaaagttatcatggccacaaaccgaatagaaactttggatccagcacttatcagaccaggccgcattgacaggaagattgagttccccctgcctgatgaaaagacgaagaagcgcatctttcagattcacacaagcaggatgacgctggctgatgatgtaaccctggacgacctgatcatggctaaagatgacctctctggtgctgacatcaaggcaatctgtacagaagctggtctgatggccttaagagaacgtagaatgaaagtaacaaatgaagacttcaaaaaatctaaagaaaatgttctttataagaaacaggaaggcacccctgaggggctgtatctc

SEQ ID NO: 17 - protein, 359 aa, Homo Sapiens
MSMNNSKQLVSPAAALLSNTTCQTENRLSVFFSVIFMTVGILSNSLAIAILMKAYQRFRQKSKASFLLLASGLVITDFFGHLINGAIAVFVYASDKEWIRFDQSNVLCSIFGICMVFSGLCPLLLGSVMAIERCIGVTKPIFHSTKITSKHVKMMLSGVCLFAVFIALLPILGHRDYKIQASRTWCFYNTEDIKDWEDRFYLLLFSFLGLLALGVSLLCNAITGITLLRVKFKSQQHRQGRSHHLEMVIQLLAIMCVSCICWSPFLVTMANIGINGNHSLETCETTLFALRMATWNQILDPWVYILLRKAVLKNLYKLASQCCGVHVISLHIWELSSIKNSLKVAAISESPVAEKSAST

SEQ ID NO: 18 - DNA, 1077 bp, Homo Sapiens
atgtccatgaacaattccaaacagctagtgtctcctgcagctgcgcttctttcaaacacaacctgccagacggaaaaccggctttccgtatttttttcagtaatcttcatgacagtgggaatcttgtcaaacagccttgccatcgccattctcatgaaggcatatcagagatttagacagaagtccaaggcatcgtttctgcttttggccagtggcctggtaatcactgatttctttggccatctcatcaatggagccatagcagtatttgtatatgcttctgataaagaatggatccgctttgaccaatcaaatgtcctttgcagtatttttggtatctgcatggtgttttctggtctgtgcccacttcttctaggcagtgtgatggccattgagcggtgtattggagtcacaaaaccaatatttcattctacgaaaattacatccaaacatgtgaaaatgatgttaagtggtgtgtgcttgtttgctgttttcatagctttgctgcccatccttggacatcgagactataaaattcaggcgtcgaggacctggtgtttctacaacacagaagacatcaaagactgggaagatagattttatcttctacttttttcttttctggggctcttagcccttggtgtttcattgttgtgcaatgcaatcacaggaattacacttttaagagttaaatttaaaagtcagcagcacagacaaggcagatctcatcatttggaaatggtaatccagctcctggcgataatgtgtgtctcctgtatttgttggagcccatttctggttacaatggccaacattggaataaatggaaatcattctctggaaacctgtgaaacaacactttttgctctccgaatggcaacatggaatcaaatcttagatccttgggtatatattcttctacgaaaggctgtccttaagaatctctataagcttgccagtcaatgctgtggagtgcatgtcatcagcttacatatttgggagcttagttccattaaaaattccttaaaggttgctgctatttctgagtcaccagttgcagagaaatcagcaagcacc

SEQ ID NO: 19 - protein, 793 aa, Homo Sapiens
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETPIIAVMVALSSLLVIVFIIIVLYMLRFKKYKQAGSHSNSFRLSNGRTEDVEPQSVPLLARSPSTNRKYPPLPVDKLEEEINRRMADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKECKCAQYWPDQGCWTYGNIRVSVEDVTVLVDYTVRKFCIQQVGDMTNRKPQRLITQFHFTSWPDFGVPFTPIGMLKFLKKVKACNPQYAGAIVVHCSAGVGRTGTFVVIDAMLDMMHTERKVDVYGFVSRIRAQRCQMVQTDMQYVFIYQALLEHYLYGDTELEVTSLETHLQKIYNKIPGTSNNGLEEEFKKLTSIKIQNDKMRTGNLPANMKKNRVLQIIPYEFNRVIIPVKRGEENTDYVNASFIDGYRQKDSYIASQGPLLHTIEDFWRMIWEWKSCSIVMLTELEERGQEKCAQYWPSDGLVSYGDITVELKKEEECESYTVRDLLVTNTRENKSRQIRQFHFHGWPEVGIPSDGKGMISIIAAVQKQQQQSGNHPITVHCSAGAGRTGTFCALSTVLERVKAEGILDVFQTVKSLRLQRPHMVQTLEQYEFCYKVVQEYIDAFSDYANFK

SEQ ID NO: 20 - DNA, 2379 bp, Homo Sapiens
atggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgacaataagctcttcagggaggaattcaacgctctccctgcatgtcctatccaggccacctgtgaggctgcttccaaggaggaaaacaaggaaaaaaatcgatatgtaaacatcttgccttatgaccactctagagtccacctgacaccggttgaaggggttccagattctgattacatcaatgcttcattcatcaacggttaccaagaaaagaacaaattcattgctgcacaaggaccaaaagaagaaacggtgaatgatttctggcggatgatctgggaacaaaacacagccaccatcgtcatggttaccaacctgaaggagagaaaggagtgcaagtgcgcccagtactggccagaccaaggctgctggacctatgggaatattcgggtgtctgtagaggatgtgactgtcctggtggactacacagtacggaagttctgcatccagcaggtgggcgacatgaccaacagaaagccacagcgcctcatcactcagttccactttaccagctggccagactttggggtgccttttaccccgatcggcatgctcaagttcctcaagaaggtgaaggcctgtaaccctcagtatgcaggggccatcgtggtccactgcagtgcaggtgtagggcgtacaggtacctttgtcgtcattgatgccatgctggacatgatgcatacagaacggaaggtggacgtgtatggctttgtgagccggatccgggcacagcgctgccagatggtgcaaaccgatatgcagtatgtcttcatataccaagcccttctggagcattatctctatggagatacagaactggaagtgacctctctagaaacccacctgcagaaaatttacaacaaaatcccagggaccagcaacaatggattagaggaggagtttaagaagttaacatcaatcaaaatccagaatgacaagatgcggactggaaaccttccagccaacatgaagaagaaccgtgttttacagatcattccatatgaattcaacagagtgatcattccagttaagcggggcgaagagaatacagactatgtgaacgcatcctttattgatggctaccggcagaaggactcctatatcgccagccagggccctcttctccacacaattgaggacttctggcgaatgatctgggagtggaaatcctgctctatcgtgatgctaacagaactggaggagagaggccaggagaagtgtgcccagtactggccatctgatggactggtgtcctatggagatattacagtggaactgaagaaggaggaggaatgtgagagctacaccgtccgagacctcctggtcaccaacaccagggagaataagagccggcagatccggcagttccacttccatggctggcctgaagtgggcatccccagtgacggaaagggcatgatcagcatcatcgccgccgtgcagaagcagcagcagcagtcagggaaccaccccatcaccgtgcactgcagcgccggggcaggaaggacggggaccttctgtgccctgagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaag

SEQ ID NO: 21 - protein, 203 aa, Homo sapiens
MGSRDHLFKVLVVGDAAVGKTSLVQRYSQDSFSKHYKSTVGVDFALKVLQWSDYEIVRLQLWDIAGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 22 - DNA, 609 bp, Homo sapiens
atgggcagccgcgaccacctgttcaaagtgctggtggtgggggacgccgcagtgggcaagacgtcgctggtgcagcgatattcccaggacagcttcagcaaacactacaagtccacggtgggagtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgc

SEQ ID NO: 23 - protein, 688 aa, Homo sapiens
MGSENSALKSYTLREPPFTLPSGLAVYPAVLQDGKFASVFVYKRENEDKVNKAAKHLKTLRHPCLLRFLSCTVEADGIHLVTERVQPLEVALETLSSAEVCAGIYDILLALIFLHDRGHLTHNNVCLSSVFVSEDGHWKLGGMETVCKVSQATPEFLRSIQSIRDPASIPPEEMSPEFTTLPECHGHARDAFSFGTLVESLLTILNEQVSADVLSSFQQTLHSTLLNPIPKCRPALCTLLSHDFFRNDFLEVVNFLKSLTLKSEEEKTEFFKFLLDRVSCLSEELIASRLVPLLLNQLVFAEPVAVKSFLPYLLGPKKDHAQGETPCLLSPALFQSRVIPVLLQLFEVHEEHVRMVLLSHIEAYVEHFTQEQLKKVILPQVLLGLRDTSDSIVAITLHSLAVLVSLLGPEVVVGGERTKIFKRTAPSFTKNTDLSLEGDPFSQPIKFPINGLSDVKNTSEDSENFPSSSKKSEEWPDWSEPEEPENQTVNIQIWPREPCDDVKSQCTTLDVEESSWDDCEPSSLDTKVNPGGGITATKPVTSAEQKPIPALLSLTEESMPWKSSLPQKISLVQRGDDADQIEPPKVSSQERPLKVPSELGLGEEFTIQVKKKPVKDPEMDWFADMIPEIKPSAAFLILPELRTEMVPKKDDVSPVMQFSSKFAAAEITEGEAEGWEEEGELNWEDNNW

SEQ ID NO: 24 - DNA, 2064 bp, Homo sapiens
atgggatcagagaacagtgctttaaagagctatacactgagagaaccaccatttaccttaccctctggacttgctgtttatcccgctgtactgcaagatggcaaatttgcttcagtttttgtgtataagagagaaaatgaagacaaggttaataaagctgccaagcatttgaagacacttcgtcacccttgcttgctaagatttttatcttgtactgtggaagcggatggcattcatcttgtcactgagcgagtacagcccctggaagtggctttggaaacattgtcttctgcagaggtctgtgctgggatctatgacatattgctggctcttatcttccttcatgacagaggacacctaacacacaataatgtctgtttatcatctgtgtttgtgagtgaagatggacactggaagctaggaggaatggaaactgtttgtaaagtttctcaggccacaccagagtttctgaggagtattcagtcaataagagacccagcatctatccctcctgaagagatgtctccagaattcacaactctcccagagtgtcatggacatgcccgggatgccttttcatttggaacattggtggaaagtttgctcacaatcttaaatgaacaggtttcagcggatgttctctccagctttcaacagaccttgcactcaactttgctgaatcccattccaaaatgtcggccagcgctctgcaccttactatctcatgacttcttcagaaatgattttctggaagttgtgaatttcttgaaaagtttaacattgaagagtgaagaggagaaaacggaattctttaaatttctgctggacagagtcagctgcttgtcagaggaattgatagcttcaaggttggtgcctcttctgcttaatcagttggtgtttgcagagccagtggctgttaagagttttcttccttatctgcttggccccaaaaaagatcatgcgcagggagaaactccttgcttgctctcaccagccctgttccagtcacgggtgatccccgtgcttctccagttgtttgaagttcatgaagagcatgtgcggatggtgctgctgtctcacatcgaggcctacgtggagcacttcactcaggagcagctgaagaaagtcatcttgccacaggttttgctgggcctgcgtgatactagtgattccattgtggcaattactctgcatagcctagcagtgctggtctctctgcttggaccagaggtggttgtgggaggagaacgaaccaagatcttcaaacgcactgccccaagttttactaaaaatactgacctttctctagaaggtgatccattttctcagcctattaaatttcccataaacggactctcagatgtaaaaaatacttcggaggacagtgaaaacttcccatcaagttctaaaaagtctgaggagtggcctgactggagtgaacctgaggagcctgaaaatcaaactgtcaacatacagatttggcctagagaaccttgtgatgatgtcaagtcccagtgcactaccttggatgtggaagagtcatcttgggatgactgcgagcccagcagcttagatactaaagtaaacccaggaggtggaatcactgctacaaaacctgttacctcagcggagcagaagcctattcctgctttgctttcactcactgaagagtctatgccttggaaatcaagcttaccccaaaagattagccttgtacaaaggggggatgacgcagaccaaatcgagccgccaaaagtgtcatcacaagaaaggccccttaaggttccatcagaacttggtttaggagaggaattcaccattcaagtaaaaaagaagccagtaaaagatcctgagatggattggtttgctgatatgatcccagaaattaagccttctgctgcttttcttatattacctgaactgaggacagaaatggtcccaaaaaaggatgatgtctccccagtgatgcagttttcctcaaaatttgctgcagcagaaattactgagggagaggctgaaggctgggaagaagaaggggagctgaactgggaagataataactgg

(19) United States
(12) Patent Application Publication
(10) Pub. No.: US 2015/0362497 A1
(43) Pub. Date: Dec. 17, 2015

(54) Autoantibody Signature for the Early Detection of Ovarian Cancer

(71) Applicants: Karen ANDERSON, Scottsdlae, AZ (US); Joshua LABAER, Chandler, AZ (US); Garrick WALLSTROM, Mesa, AZ (US); Daniel CRAMER, Chestnut Hill, MA (US); ARIZONA BOARD OF REGENTS, A BODY CORPRATE OF THE S TATE OF ARIZONA, ACTING FOR AND BEHALF OF ARI, Scottsdale, AZ (US)

(72) Inventors: Karen Anderson, Scottsdale, AZ (US); Joshua LaBaer, Chandler, AZ (US); Garrick Wallstrom, Mesa, AZ (US); Daniel Cramer, Chestnut Hill, AZ (US)

(73) Assignee: Arizona Board of Regents on behalf of Arizona Stat State University, Scottsdale, AZ (US)

(21) Appl. No.: 14/763,492

(22) PCT Filed: Jan. 30, 2014
(86) PCT No.: PCT/US2014/013809

§ 371 Date: Jul. 24, 2015

Related U.S. Application Data
(60) Provisional application No. 61/759,047, filed on Jan. 31, 2013.

(51) Int. Cl. G01N 33/574

(52) U.S. Cl. CPC G01N 33/57488; G01N 2800/7028; G01N 2333/4748; G01N 2333/5756; G01N 2333/471; G01N 2333/90666; G01N 2333/914

(57) ABSTRACT

Methods for identifying antigens as potential biomarkers for the early detection of ovarian cancer, as well as kits for utilizing said antigens as biomarkers and in personalized medicine/therapeutics assessment. Protein microarrays displaying full-length candidate antigens were developed and sequentially screening to select candidate autoantibody biomarkers to limit the false discovery rate inherent to large-scale proteomic screening.

### CROSS-REFERENCE TO RELATED APPLICATION

This application claims priority to U.S. provisional patent Application No. 61/759,047 filed on Jan. 31, 2013, which is incorporated by reference herein in its entirety.

### STATEMENT REGARDING FEDERALLY SPONSORED RESEARCH OR DEVELOPMENT

This invention was made with government support under CA117374 awarded by The National Institutes of Health. The U.S. government has certain rights in the invention.

### BACKGROUND OF THE INVENTION

Ovarian cancer is the fifth leading cause of cancer-related mortality of women in the U.S., with over 15,000 deaths per year. Early diagnosis is associated with improved overall survival; however, the majority of patients are currently diagnosed with advanced disease. The five-year survival rate for late-stage ovarian cancer remains less than 30%.

Despite the identification of serum CA 125 as a biomarker for ovarian cancer in 1983, there are currently no screening biomarkers recommended for use for the general population. The utility of CA 125 as a screening test is limited by a low sensitivity of 50% for early stage disease at 99% specificity.

Combining CA 125 with transvaginal ultrasound (TVUS) increased the specificity of detection in the UKCTOCS large-scale screening trial. In a recent joint validation study of 28 potential markers for detecting ovarian cancer in blood, the most accurate marker remains CA 125, followed closely by HE4. Panels of markers demonstrated only marginal improvements over CA 125 alone for the early detection of disease.

A recent study showed that the addition of CEA and VCAM-1 to CA 125 and HE4 increased the sensitivity of detection of stage I and II ovarian cancer to 86% at 98% specificity, but this remains to be confirmed in a blinded validation study using prediagnostic sera.

Protein overexpression or mutation can also lead to the spontaneous development of autoantibodies (AAb) in the sera of patients with cancer. Tumor antigen-specific AAb have been identified in the sera of patients with cancer, including patients with early-stage disease. p53-specific AAb, which are associated with p53 mutation and resultant protein stabilization, have been detected in early-stage ovarian cancer. p53-specific AAb have also been detected in 41.7% of patients with serous ovarian cancer at 91.7% specificity. Unlike CA 125 and HE4, p53-AAb were associated with improved survival.

However, identification and utilization of other biomarkers for detection of early stage ovarian cancer remains elusive.

### SUMMARY OF THE INVENTION

Methods to identify antibody signatures in early-stage breast cancer using Nucleic Acid Protein Programmable Arrays (NAPPA) have been developed. Diagnostic test kits and personalized medicine determinations, such as the identification of biomarkers for the early detection of ovarian cancer, also are disclosed.

A novel protein microarray technology NAPPA, which are generated by printing full-length cDNAs encoding the target proteins at each feature of the array, was used. The proteins are then transcribed and translated by a cell-free system and immobilized in situ using epitope tags fused to the proteins. Sera are added, and bound IgG is detected by standard secondary reagents.

These and other aspects of the invention will be apparent upon reference to the following detailed description and figures. To that end, any patent and other documents cited herein are hereby incorporated by reference in their entirety.

### DETAILED DESCRIPTION OF THE INVENTION

Embodiments described herein relate to methods for identifying autoantibodies as potential biomarkers for the early detection of ovarian cancer, as well as to kits for utilizing said autoantibodies as diagnostic biomarkers and for personalized medicine/therapeutics assessment.

Protein microarrays displaying full-length candidate antigens have been developed and sequentially screened to select candidate autoantibody biomarkers. Sera from patients with ovarian cancer were found to contain autoantibodies (AAb) to tumor-derived proteins. Thus, to detect AAb, high-density programmable protein microarrays (NAPPA) expressing 5,177 candidate tumor antigens are probed with sera from patients with serous ovarian cancer and healthy controls, bound IgG measured.

In one embodiment, a set of 741 antigens was selected and probed with an independent set of sera from serous ovarian cancer patients and matched controls. Twelve potential autoantigens were identified with sensitivities ranging from 13-22% at >93% specificity. Surprisingly, many of these twelve autoantigens are not known to previously have been associated with ovarian cancer.

### Non-Limiting Examples

The objective of this study was to identify novel AAb biomarkers for the detection of serous ovarian cancer. To profile the ovarian cancer immune response, NAPPA microarrays displaying 5,177 full-length candidate antigens were generated using cDNAs derived from the DNASU Plasmid Repository at Arizona State University. These cDNAs were all sequence-verified, full length, wild-type genes fused in frame with either a C-terminal GST tag or N-terminal FLAG tag in a vector optimized for mammalian protein expression.

The cDNAs were printed on amine-treated glass slides with anti-tag antibodies at a high density (up to 2300 antigens/slide; 3 slides/gene set) using a Genetix QArray2 with 300 pm solid tungsten pins. Proteins were expressed and captured in situ on the arrays using a coupled in vitro transcription-translation system derived from rabbit reticulocyte lysate. Protein expression was confirmed by probing the arrays with anti-tag antibodies. For detecting antibodies, the arrays were incubated with serum diluted in 5% PBS mile with 0.2% Tween 20 overnight and detected with anti-human IgG-HRP with Tyramide. Slides were scanned with a Perkin Elmer ProScanArray HT and the images quantitated using ArrayPro software.

A sequential screening strategy was used to select candidate AAb biomarkers to limit the false discovery rate inherent to large-scale proteomic screening.

First, 34 cases of serous ovarian cancer and 30 age-matched healthy controls (Cohort 1) were screened on 5,177 candidate tumor antigens. Each array was normalized by first removing the background signal estimated by the first quartile of the non-spots and then log-transforming the median-scaled raw intensities to bring the data to the same scale and stabilize the variance across the range of signals.

Candidate antigens from the initial 5,177 antigens were selected if they met two different criteria: 1) comparison of the 95^{th} percentiles of the cases and controls using quantile regression and 2) comparison of the proportion of cases with intensities above the 95^{th} percentile of controls to the expected number seen by chance using binomial tests. Using these criteria, 741 antigens were selected for further testing.

Second, 60 cases and 60 healthy controls (Cohort 2) were screened on the 741 antigens, which were printed in duplicate on the arrays. Following the use of spot-level quality control procedures, the arrays were similarly normalized and analyzed using receiver operator characteristic (ROC) curve analysis. Specifically, we tested the hypothesis that the partial area under the ROC curve (PAUC) in the region where the specificity >95% exceeds 0.00125, which is the PAUC for a non-informative diagnostic test. We computed q-values and identified 12 potential AAb biomarkers that were statistically significant with a false discovery rate threshold of 15% (Table 1).

Descriptions of these biomarkers, their amino acid sequences and their nucleic acid sequences are provided in Table 2. Third, an independent assay (Luminex bead array) was used to display these autoantigens, and sera from women in Cohort 2 was re-screened. Finally, a smaller set of 7 autoantigens was displayed and screened with sera from an independent set (Cohort 3) of non-serous cancers (n=30), false-negative CA 125 (n=20), benign ovarian disease (n=30), and healthy controls (n=30).

TABLE 1
NAPPA Results for 12 Potential Biomarkers for Ovarian Cancer

| Protein | PAUC P-Value | Sensitivity | Specificity |
|---|---|---|---|
| ACSBG1 | 0.0020 | 15.5% | 93.3% |
| AFP | 0.0040 | 15.5% | 93.3% |
| CSNK1A1L | 0.0047 | 13.8% | 93.3% |
| DHFR | 0.0057 | 17.2% | 93.3% |
| MBNL1 | 0.0067 | 13.8% | 93.3% |
| p53 | 0.0067 | 21.4% | 93.1% |
| PRL | 0.0026 | 17.2% | 93.3% |
| PSMC1 | 0.0057 | 15.5% | 93.3% |
| PTGFR | 0.0070 | 15.5% | 93.3% |
| PTPRA | 0.0004 | 22.4% | 93.3% |
| RAB7L1 | 0.0034 | 20.7% | 93.3% |
| SCYL3 | 0.0063 | 19.0% | 93.3 |

The twelve biomarkers for ovarian cancer can be utilized on an array or other substrate as a diagnostic test in which sera from a patient is tested for ovarian cancer autoantibodies.

TABLE 2
Candidate Biomarkers

ACSBG1 NP_055977 acyl-CoA synthetase bubblegum family member 1 full
length (1-724), M633V
Amino acid sequence
(SEQ ID NO: 1)
MPRNSGAGYGCPHGDPSMLDSRETPQESRQDMIVRTTQEKLKTSSLTDRQPLSKESLNHALELSVPEKVNN
AQWDAPEEALWTTRADGRVRLRIDPSCPQLPYTVHRMFYEALDKYGDLIALGFKRQDKWEHISYSQYYLLA
RRAAKGFLKLGLKQAHSVAILGFNSPEWFFSAVGTVFAGGIVTGIYTTSSPEACQYIAYDCCANVIMVDTQ
KQLEKILKIWKQLPHLKAVVIYKEPPPNKMANVYTMEEFMELGNEVPEEALDAIIDTQQPNQCCVLVYTSG
TTGNPKGVMLSQDNITWTARYGSQAGDIRPAEVQQEVVVSYLPLSHIAAQIYDLWTGIQWGAQVCFAEPDA
LKGSLVNTLREVEPTSHMGVPRVWEKIMERIQEVAAQSGFIRRKMLLWAMSVTLEQNLTCPGSDLKPFTTR
LADYLVLAKVRQALGFAKCQKNFYGAAPMMAETQHFFLGLNIRLYAGYGLSETSGPHFMSSPYNYRLYSSG
KLVPGCRVKLVNQDAEGIGEICLWGRTIFMGYLNMEDKTCEAIDEEGWLHTGDAGRLDADGFLYITGRLKE
LIITAGGENVPPVPIEEAVKMELPIISNAMLIGDQRKFLSMLLTLKCTLDPDTSDQTDNLTEQAVEFCQRV
GSRATTVSEIIEKKDEAVYQAIEEGIRRVNMNAAARPYHIQKWAILERDFSISGGELGPTMKLKRLTVLEK
YKGIIDSFYQEQKM
Encoding nucleotide sequence
(SEQ ID NO: 2)
ATGCCACGCAATTCTGGAGCTGGATACGGCTGCCCACACGGGGACCCCAGCATGCTGGACAGCAGAGAGAC
CCCACAGGAGAGCCGGCAGGACATGATTGTGAGGACCACCCAAGAAAAATTGAAAACCAGCTCACTGACTG
ACAGGCAGCCACTCTCCAAAGAGTCCCTGAACCATGCTCTCGAGCTCTCAGTGCCAGAGAAGGTGAATAAT
GCCCAGTGGGATGCTCCAGAGGAGGCGCTGTGGACGACTCGGGCCGATGGGCGGGTGCGCCTGCGCATAGA
CCCCAGCTGCCCACAGCTTCCCTACACTGTGCATCGGATGTTCTACGAGGCCCTGGATAAGTATGGGGACC
TCATCGCTTTGGGCTTCAAGCGCCAGGACAAGTGGGAACACATCTCCTACTCCCAATACTACCTGCTCGCC
CGCAGAGCCGCCAAGGGCTTCCTGAAGCTCGGCCTGAAGCAGGCCCACAGTGTGGCCATCCTCGGCTTCAA
CTCCCCGGAGTGGTTCTTCTCGGCAGTGGGCACAGTATTTGCAGGTGGCATCGTCACTGGCATCTACACCA
CCAGCTCCCCAGAGGCCTGCCAGTACATCGCTTATGACTGCTGCGCCAATGTCATCATGGTCGACACGCAG
AAGCAGCTGGAAAAGATCCTGAAGATCTGGAAACAGTTGCCACATCTAAAGGCAGTCGTGATATATAAAGA
ACCTCCTCCAAACAAGATGGCCAATGTGTACACGATGGAGGAATTCATGGAGCTGGGGAATGAAGTGCCTG
AGGAAGCCCTGGACGCCATCATTGACACCCAGCAGCCCAACCAGTGCTGTGTGCTAGTCTACACTTCCGGC
ACCACTGGGAACCCCAAGGGCGTGATGCTGAGTCAAGACAATATCACGTGGACGGCACGGTACGGCAGCCA
GGCCGGTGACATCCGGCCGGCAGAAGTCCAGCAGGAGGTGGTAGTCAGCTACCTGCCCCTCAGCCATATTG
CCGCCCAGATCTACGACCTGTGGACAGGCATCCAGTGGGGGGCCCAGGTTTGCTTTGCCGAACCCGACGCC
CTGAAGGGGAGCCTGGTGAACACGCTGCGGGAGGTGGAGCCCACATCACACATGGGGGTGCCCCGGGTATG
GGAGAAGATCATGGAGCGCATCCAGGAGGTGGCGGCTCAGTCTGGCTTCATCCGGCGGAAGATGCTGCTGT
GGGCCATGTCGGTGACCTTGGAGCAGAACCTCACCTGCCCCGGCAGCGACCTGAAGCCCTTCACAACCAGA
CTGGCAGATTACCTGGTGCTAGCCAAGGTTCGCCAGGCACTGGGATTTGCCAAGTGTCAAAAGAACTTCTA
TGGAGCGGCCCCCATGATGGCAGAGACACAGCACTTCTTCCTGGGTCTCAACATCCGCTTGTATGCGGGCT
ATGGCCTCAGTGAGACCTCAGGCCCCCACTTCATGTCCAGTCCCTACAACTACCGGCTGTACAGCTCAGGC
AAGTTGGTGCCCGGCTGTCGGGTGAAGCTGGTGAACCAGGACGCAGAGGGCATTGGTGAGATCTGCCTGTG
GGGCCGCACCATATTCATGGGCTACCTGAACATGGAGGACAAGACTTGTGAGGCCATCGACGAGGAAGGCT
GGCTGCACACGGGTGATGCTGGCCGCCTGGACGCCGATGGCTTCCTCTACATCACTGGGCGCCTCAAAGAA
TTAATCATCACAGCTGGTGGGGAGAATGTGCCCCCTGTGCCCATCGAGGAGGCCGTGAAGATGGAGCTGCC
CATCATCAGCAATGCCATGCTCATTGGGGACCAGAGGAAGTTCCTGTCCATGCTGCTCACCTTGAAGTGCA
CTCTGGACCCAGACACCTCTGACCAGACTGATAATCTGACTGAACAAGCTGTGGAGTTCTGCCAGAGGGTG
GGCAGCAGAGCCACCACAGTGTCCGAGATCATAGAGAAGAAGGATGAGGCCGTGTACCAGGCCATCGAAGA
GGGGATCCGGAGGGTCAACATGAACGCGGCGGCCCGGCCCTACCACATCCAGAAGTGGGCCATTCTCGAGA
GAGACTTCTCCATTTCGGGTGGAGAGTTGGGTCCCACGATGAAACTGAAACGGCTCACAGTTTTGGAGAAG
TACAAAGGTATCATTGACTCCTTTTACCAAGAGCAAAAAATG
AFP NP_001125 alpha-fetoprotein full length (1-609)
Amino acid sequence
(SEQ ID NO: 3)
MKWVESIFLIFLLNFTESRTLHRNEYGIASILDSYQCTAEISLADLATIFFAQFVQEATYKEVSKMVKDAL
TAIEKPTGDEQSSGCLENQLPAFLEELCHEKEILEKYGHSDCCSQSEEGRHNCFLAHKKPTPASIPLFQVP
EPVTSCEAYEEDRETFMNKFIYEIARRHPFLYAPTILLWAARYDKIIPSCCKAENAVECFQTKAATVTKEL
RESSLLNQHACAVMKNFGTRTFQAITVTKLSQKFTKVNFTEIQKLVLDVAHVHEHCCRGDVLDCLQDGEKI
MSYICSQQDTLSNKITECCKLTTLERGQCIIHAENDEKPEGLSPNLNRFLGDRDFNQFSSGEKNIFLASFV
HEYSRRHPQLAVSVILRVAKGYQELLEKCFQTENPLECQDKGEEELQKYIQESQALAKRSCGLFQKLGEYY
LQNAFLVAYTKKAPQLTSSELMAITRKMAATAATCCQLSEDKLLACGEGAADIIIGHLCIRHEMTPVNPGV
GQCCTSSYANRRPCFSSLVVDETYVPPAFSDDKFIFHKDLCQAQGVALQTMKQEFLINLVKQKPQITEEQL
EAVIADFSGLLEKCCQGQEQEVCFAEEGQKLISKTRAALGV
Encoding nucleotide sequence
(SEQ ID NO: 4)
ATGAAGTGGGTGGAATCAATTTTTTTAATTTTCCTACTAAATTTTACTGAATCCAGAACACTGCATAGAAA
TGAATATGGAATAGCTTCCATATTGGATTCTTACCAATGTACTGCAGAGATAAGTTTAGCTGACCTGGCTA
CCATATTTTTTGCCCAGTTTGTTCAAGAAGCCACTTACAAGGAAGTAAGCAAAATGGTGAAAGATGCATTG
ACTGCAATTGAGAAACCCACTGGAGATGAACAGTCTTCAGGGTGTTTAGAAAACCAGCTACCTGCCTTTCT
GGAAGAACTTTGCCATGAGAAAGAAATTTTGGAGAAGTACGGACATTCAGACTGCTGCAGCCAAAGTGAAG
AGGGAAGACATAACTGTTTTCTTGCACACAAAAAGCCCACTCCAGCATCGATCCCACTTTTCCAAGTTCCA
GAACCTGTCACAAGCTGTGAAGCATATGAAGAAGACAGGGAGACATTCATGAACAAATTCATTTATGAGAT
AGCAAGAAGGCATCCCTTCCTGTATGCACCTACAATTCTTCTTTGGGCTGCTCGCTATGACAAAATAATTC
CATCTTGCTGCAAAGCTGAAAATGCAGTTGAATGCTTCCAAACAAAGGCAGCAACAGTTACAAAAGAATTA
AGAGAAAGCAGCTTGTTAAATCAACATGCATGTGCAGTAATGAAAAATTTTGGGACCCGAACTTTCCAAGC
CATAACTGTTACTAAACTGAGTCAGAAGTTTACCAAAGTTAATTTTACTGAAATCCAGAAACTAGTCCTGG
ATGTGGCCCATGTACATGAGCACTGTTGCAGAGGAGATGTGCTGGATTGTCTGCAGGATGGGGAAAAAATC
ATGTCCTACATATGTTCTCAACAAGACACTCTGTCAAACAAAATAACAGAATGCTGCAAACTGACCACGCT
GGAACGTGGTCAATGTATAATTCATGCAGAAAATGATGAAAAACCTGAAGGTCTATCTCCAAATCTAAACA
GGTTTTTAGGAGATAGAGATTTTAACCAATTTTCTTCAGGGGAAAAAAATATCTTCTTGGCAAGTTTTGTT
CATGAATATTCAAGAAGACATCCTCAGCTTGCTGTCTCAGTAATTCTAAGAGTTGCTAAAGGATACCAGGA
GTTATTGGAGAAGTGTTTCCAGACTGAAAACCCTCTTGAATGCCAAGATAAAGGAGAAGAAGAATTACAGA
AATACATCCAGGAGAGCCAAGCATTGGCAAAGCGAAGCTGCGGCCTCTTCCAGAAACTAGGAGAATATTAC
TTACAAAATGCGTTTCTCGTTGCTTACACAAAGAAAGCCCCCCAGCTGACCTCGTCGGAGCTGATGGCCAT
CACCAGAAAAATGGCAGCCACAGCAGCCACTTGTTGCCAACTCAGTGAGGACAAACTATTGGCCTGTGGCG
AGGGAGCGGCTGACATTATTATCGGACACTTATGTATCAGACATGAAATGACTCCAGTAAACCCTGGTGTT
GGCCAGTGCTGCACTTCTTCATATGCCAACAGGAGGCCATGCTTCAGCAGCTTGGTGGTGGATGAAACATA
TGTCCCTCCTGCATTCTCTGATGACAAGTTCATTTTCCATAAGGATCTGTGCCAAGCTCAGGGTGTAGCGC
TGCAAACGATGAAGCAAGAGTTTCTCATTAACCTTGTGAAGCAAAAGCCACAAATAACAGAGGAACAACTT
GAGGCTGTCATTGCAGATTTCTCAGGCCTGTTGGAGAAATGCTGCCAAGGCCAGGAACAGGAAGTCTGCTT
TGCTGAAGAGGGACAAAAACTGATTTCAAAAACTCGTGCTGCTTTGGGAGTT
CSNK1A1L NP_660204 casein kinase 1, alpha 1-like full length
(1-337), R224K
Amino acid sequence
(SEQ ID NO: 5)
MTNNSGSKAELVVGGKYKLVRKIGSGSFGDVYLGITTTNGEEVAVKLESQKVKHPQLLYESKLYTILQGGV
GIPHMHWYGQEKDNNVLVMDLLGPSLEDLFNFCSRRFTMKTVLMLADQMISRIEYVHTKNFLHRDIKPDNF
LMGTGRHCNKLFLIDFGLAKKYRDNRTRQHIPYREDKHLIGTVRYASINAHLGIEQSRRDDMESLGYVFMY
FNRTSLPWQGLKAMTKKQKYEKISEKKMSTPVEVLCKGFPAEFAMYLNYCRGLRFEEVPDYMYLRQLFRIL
FRTLNHQYDYTFDWTMLKQKAAQQAASSSGQGQQAQTQTGKQTEKNKNNVKDN
Encoding nucleotide sequence
(SEQ ID NO: 6)
ATGACAAACAACAGCGGCTCCAAAGCCGAACTCGTTGTGGGAGGGAAATACAAACTGGTGCGGAAGATCGG
GTCTGGCTCCTTTGGAGACGTTTATCTGGGCATCACCACCACCAACGGCGAGGAAGTAGCAGTGAAGCTGG
AATCTCAGAAGGTCAAGCACCCCCAGTTGCTGTATGAGAGCAAACTCTACACGATTCTTCAAGGTGGGGTT
GGCATCCCCCACATGCACTGGTATGGTCAGGAAAAAGACAACAATGTGCTAGTCATGGACCTTCTGGGACC
CAGCCTCGAAGACCTCTTTAATTTCTGTTCAAGAAGGTTCACCATGAAAACTGTACTTATGTTAGCCGACC
AGATGATCAGCAGAATTGAATACGTGCATACAAAGAATTTTCTACACCGAGACATTAAACCAGATAACTTC
CTGATGGGTACTGGGCGTCACTGTAATAAGTTGTTCCTTATTGATTTTGGTTTGGCCAAAAAGTACAGAGA
CAACAGGACCAGGCAACACATACCGTACAGAGAAGATAAACACCTCATTGGCACTGTCCGATATGCCAGCA
TCAATGCACATCTTGGTATTGAGCAGAGCCGCCGAGATGACATGGAATCCTTAGGCTACGTTTTCATGTAT
TTTAATAGAACCAGCCTGCCGTGGCAAGGACTAAAGGCTATGACAAAAAAACAAAAATATGAAAAGATTAG
TGAGAAGAAGATGTCCACCCCTGTTGAAGTTTTATGTAAGGGGTTTCCTGCAGAATTCGCCATGTACTTGA
ACTACTGTCGTGGGCTGCGCTTTGAGGAAGTCCCAGATTACATGTATCTGAGGCAGCTATTCCGCATTCTT
TTCAGGACCCTGAACCACCAATATGACTACACATTTGATTGGACGATGTTAAAGCAGAAAGCAGCACAGCA
GGCAGCCTCTTCCAGTGGGCAGGGTCAGCAGGCCCAAACCCAGACAGGCAAGCAAACTGAAAAAAACAAGA
ATAATGTGAAAGATAAC
DHFR NP_000782 dihydrofolate reductase full length (1-187)
Amino acid sequence
(SEQ ID NO: 7)
MVGSLNCIVAVSQNMGIGKNGDLPWPPLRNEFRYFQRMTTTSSVEGKQNLVIMGKKTWFSIPEKNRPLKGR
INLVLSRELKEPPQGAHFLSRSLDDALKLTEQPELANKVDMVWIVGGSSVYKEAMNHPGHLKLFVTRIMQD
FESDTFFPEIDLEKYKLLPEYPGVLSDVQEEKGIKYKFEVYEKND
Encoding nucleotide sequence
(SEQ ID NO: 8)
ATGGTTGGTTCGCTAAACTGCATCGTCGCTGTGTCCCAGAACATGGGCATCGGCAAGAACGGGGACCTGCC
CTGGCCACCGCTCAGGAATGAATTCAGATATTTCCAGAGAATGACCACAACCTCTTCAGTAGAAGGTAAAC
AGAATCTGGTGATTATGGGTAAGAAGACCTGGTTCTCCATTCCTGAGAAGAATCGACCTTTAAAGGGTAGA
ATTAATTTAGTTCTCAGCAGAGAACTCAAGGAACCTCCACAAGGAGCTCATTTTCTTTCCAGAAGTCTAGA
TGATGCCTTAAAACTTACTGAACAACCAGAATTAGCAAATAAAGTAGACATGGTCTGGATAGTTGGTGGCA
GTTCTGTTTATAAGGAAGCCATGAATCACCCAGGCCATCTTAAACTATTTGTGACAAGGATCATGCAAGAC
TTTGAAAGTGACACGTTTTTTCCAGAAATTGATTTGGAGAAATATAAACTTCTGCCAGAATACCCAGGTGT
TCTCTCTGATGTCCAGGAGGAGAAAGGCATTAAGTACAAATTTGAAGTATATGAGAAGAATGAT
MBNL1 NP_066368 muscleblind-like (Drosophila) full length (1-382)
Amino acid sequence
(SEQ ID NO: 9)
MAVSVTPIRDTKWLTLEVCREFQRGTCSRPDTECKFAHPSKSCQVENGRVIACFDSLKGRCSRENCKYLHP
PPHLKTQLEINGRNNLIQQKNMAMLAQQMQLANAMMPGAPLQPVPMFSVAPSLATNASAAAFNPYLGPVSP
SLVPAEILPTAPMLVTGNPGVPVPAAAAAAAQKLMRTDRLEVCREYQRGNCNRGENDCRFAHPADSTMIDT
NDNTVTVCMDYIKGRCSREKCKYFHPPAHLQAKIKAAQYQVNQAAAAQAAATAAAMGIPQAVLPPLPKRPA
LEKTNGATAVFNTGIFQYQQALANMQLQQHTAFLPPGSILCMTPATSVVPMVHGATPATVSAATTSATSVP
FAATATANQIPIISAEHLTSHKYVTQM
Encoding nucleotide sequence
(SEQ ID NO: 10)
ATGGCTGTTAGTGTCACACCAATTCGGGACACAAAATGGCTAACACTGGAAGTATGTAGAGAGTTCCAGAG
GGGGACTTGCTCACGGCCAGACACGGAATGTAAATTTGCACATCCTTCGAAAAGCTGCCAAGTTGAAAATG
GACGAGTAATCGCCTGCTTTGATTCATTGAAAGGCCGTTGCTCCAGGGAGAACTGCAAATATCTTCATCCA
CCCCCACATTTAAAAACGCAGTTGGAGATAAATGGACGCAATAACTTGATTCAGCAGAAGAACATGGCCAT
GTTGGCCCAGCAAATGCAACTAGCCAATGCCATGATGCCTGGTGCCCCATTACAACCCGTGCCAATGTTTT
CAGTTGCACCAAGCTTAGCCACCAATGCATCAGCAGCCGCCTTTAATCCCTATCTGGGACCTGTTTCTCCA
AGCCTGGTCCCGGCAGAGATCTTGCCGACTGCACCAATGTTGGTTACAGGGAATCCGGGTGTCCCTGTACC
TGCAGCTGCTGCAGCTGCTGCACAGAAATTAATGCGAACAGACAGACTTGAGGTATGTCGAGAGTACCAAC
GTGGCAATTGCAACCGAGGAGAAAATGATTGTCGGTTTGCTCATCCTGCTGACAGCACAATGATTGACACC
AATGACAACACAGTCACTGTGTGTATGGATTACATCAAAGGGAGATGCTCTCGGGAAAAGTGCAAATACTT
TCATCCCCCTGCACATTTGCAAGCCAAGATCAAGGCTGCCCAATACCAGGTCAACCAGGCTGCAGCTGCAC
AGGCTGCAGCCACCGCAGCTGCCATGGGAATTCCTCAAGCTGTACTTCCCCCATTACCAAAGAGGCCTGCT
CTTGAAAAAACCAACGGTGCCACCGCAGTCTTTAACACTGGTATTTTCCAATACCAACAGGCTCTAGCCAA
CATGCAGTTACAACAGCATACAGCATTTCTCCCACCAGGCTCAATATTGTGCATGACACCCGCTACAAGTG
TTGTTCCCATGGTGCACGGTGCTACGCCAGCCACTGTGTCCGCAGCAACAACATCTGCCACAAGTGTTCCC
TTCGCTGCAACAGCCACAGCCAACCAGATACCCATAATATCTGCCGAACATCTGACTAGCCACAAGTATGT
TACCCAGATG
TP53 ABM86630 tumor protein p53 full length (1-393)
Amino acid sequence
(SEQ ID NO: 11)
MEEPQSDPSVEPPLSQETFSDLWKLLPENNVLSPLPSQAMDDLMLSPDDIEQWFTEDPGPDEAPRMPEAAP
RVAPAPAAPTPAAPAPAPSWPLSSSVPSQKTYQGSYGFRLGFLHSGTAKSVTCTYSPALNKMFCQLAKTCP
VQLWVDSTPPPGTRVRAMAIYKQSQHMTEVVRRCPHHERCSDSDGLAPPQHLIRVEGNLRVEYLDDRNTFR
HSVVVPYEPPEVGSDCTTIHYNYMCNSSCMGGMNRRPILTIITLEDSSGNLLGRNSFEVRVCACAGRDRRT
EEENLRKKGEPHHELPSGSTKRALPNNTSSSPQPKKKPLDGEYFTLQIRGRERFEMFRELNEALELKDAQA
GKEPGGSRAHSSHLKSKKGQSTSRHKKLMFKTEGPDSD
Encoding nucleotide sequence
(SEQ ID NO: 12)
ATGGAGGAGCCGCAGTCAGATCCTAGCGTCGAGCCCCCTCTGAGTCAGGAAACATTTTCAGACCTATGGAA
ACTACTTCCTGAAAACAACGTTCTGTCCCCCTTGCCGTCCCAAGCAATGGATGATTTGATGCTGTCCCCGG
ACGATATTGAACAATGGTTCACTGAAGACCCAGGTCCAGATGAAGCTCCCAGAATGCCAGAGGCTGCTCCC
CGCGTGGCCCCTGCACCAGCAGCTCCTACACCGGCGGCCCCTGCACCAGCCCCCTCCTGGCCCCTGTCATC
TTCTGTCCCTTCCCAGAAAACCTACCAGGGCAGCTACGGTTTCCGTCTGGGCTTCTTGCATTCTGGGACAG
CCAAGTCTGTGACTTGCACGTACTCCCCTGCCCTCAACAAGATGTTTTGCCAACTGGCCAAGACCTGCCCT
GTGCAGCTGTGGGTTGATTCCACACCCCCGCCCGGCACCCGCGTCCGCGCCATGGCCATCTACAAGCAGTC
ACAGCACATGACGGAGGTTGTGAGGCGCTGCCCCCACCATGAGCGCTGCTCAGATAGCGATGGTCTGGCCC
CTCCTCAGCATCTTATCCGAGTGGAAGGAAATTTGCGTGTGGAGTATTTGGATGACAGAAACACTTTTCGA
CATAGTGTGGTGGTGCCCTATGAGCCGCCTGAGGTTGGCTCTGACTGTACCACCATCCACTACAACTACAT
GTGTAACAGTTCCTGCATGGGCGGCATGAACCGGAGGCCCATCCTCACCATCATCACACTGGAAGACTCCA
GTGGTAATCTACTGGGACGGAACAGCTTTGAGGTGCGTGTTTGTGCCTGTGCTGGGAGAGACCGGCGCACA
GAGGAAGAGAATCTCCGCAAGAAAGGGGAGCCTCACCACGAGCTGCCCTCAGGGAGCACTAAGCGAGCACT
GCCCAACAACACCAGCTCCTCTCCCCAGCCAAAGAAGAAACCACTGGATGGAGAATATTTCACCCTTCAGA
TCCGTGGGCGTGAGCGCTTCGAGATGTTCCGAGAGCTGAATGAGGCCTTGGAACTCAAGGATGCCCAGGCT
GGGAAGGAGCCAGGGGGGAGCAGGGCTCACTCCAGCCACCTGAAGTCCAAAAAGGGTCAGTCTACCTCCCG
CCATAAAAAACTCATGTTCAAGACAGAAGGGCCTGACTCAGAC
PRL NP_000939 prolactin full length (1-227)
Amino acid sequence
(SEQ ID NO: 13)
MNIKGSPWKGSLLLLLVSNLLLCQSVAPLPICPGGAARCQVTLRDLFDRAVVLSHYIHNLSSEMFSEFDKR
YTHGRGFITKAINSCHTSSLATPEDKEQAQQMNQKDFLSLIVSILRSWNEPLYHLVTEVRGMQEAPEAILS
KAVEIEEQTKRLLEGMELIVSQVHPETKENEIYPVWSGLPSLQMADEESRLSAYYNLLHCLRRDSHKIDNY
LKLLKCRIIHNNNC
Encoding nucleotide sequence
(SEQ ID NO: 14)
ATGAACATCAAAGGATCGCCATGGAAAGGGTCCCTCCTGCTGCTGCTGGTGTCAAACCTGCTCCTGTGCCA
GAGCGTGGCCCCCTTGCCCATCTGTCCCGGCGGGGCTGCCCGATGCCAGGTGACCCTTCGAGACCTGTTTG
ACCGCGCCGTCGTCCTGTCCCACTACATCCATAACCTCTCCTCAGAAATGTTCAGCGAATTCGATAAACGG
TATACCCATGGCCGGGGGTTCATTACCAAGGCCATCAACAGCTGCCACACTTCTTCCCTTGCCACCCCCGA
AGACAAGGAGCAAGCCCAACAGATGAATCAAAAAGACTTTCTGAGCCTGATAGTCAGCATATTGCGATCCT
GGAATGAGCCTCTGTATCATCTGGTCACGGAAGTACGTGGTATGCAAGAAGCCCCGGAGGCTATCCTATCC
AAAGCTGTAGAGATTGAGGAGCAAACCAAACGGCTTCTAGAGGGCATGGAGCTGATAGTCAGCCAGGTTCA
TCCTGAAACCAAAGAAAATGAGATCTACCCTGTCTGGTCGGGACTTCCATCCCTGCAGATGGCTGATGAAG
AGTCTCGCCTTTCTGCTTATTATAACCTGCTCCACTGCCTACGCAGGGATTCACATAAAATCGACAATTAT
CTCAAGCTCCTGAAGTGCCGAATCATCCACAACAACAACTGCTTG
PSMC1 NP_032973 proteasome (prosome, macropain) 26S subunit, ATPase,
1 full length (1-440)
Amino acid sequence
(SEQ ID NO: 15)
MGQSQSGGHGPGGGKKDDKDKKKKYEPPVPTRVGKKKKKTKGPDAASKLPLVTPHTQCRLKLLKLERIKDY
LLMEEEFIRNQEQMKPLEEKQEEERSKVDDLRGTPMSVGTLEEIIDDNHAIVSTSVGSEHYVSILSFVDKD
LLEPGCSVLLNHKVHAVIGVLMDDTDPLVTVMKVEKAPQETYADIGGLDNQIQEIKESVELPLTHPEYYEE
MGIKPPKGVILYGPPGTGKTLLAKAVANQTSATFLRVVGSELIQKYLGDGPKLVRELFRVAEEHAPSIVFI
DEIDAIGTKRYDSNSGGEREIQRTMLELLNQLDGFDSRGDVKVIMATNRIETLDPALIRPGRIDRKIEFPL
PDEKTKKRIFQIHTSRMTLADDVTLDDLIMAKDDLSGADIKAICTEAGLMALRERRMKVTNEDFKKSKENV
LYKKQEGTPEGLYL
Encoding nucleotide sequence
(SEQ ID NO: 16)
ATGGGTCAAAGTCAGAGTGGTGGTCATGGTCCTGGAGGTGGCAAGAAGGATGACAAGGACAAGAAAAAGAA
ATATGAACCTCCTGTACCAACTAGAGTGGGGAAAAAGAAGAAGAAAACAAAGGGACCAGATGCTGCCAGCA
AACTGCCACTGGTGACACCTCACACTCAGTGCCGGTTAAAATTACTGAAGTTAGAGAGAATTAAAGACTAT
CTTCTCATGGAGGAAGAATTCATTAGAAATCAGGAACAAATGAAACCATTAGAAGAAAAGCAAGAGGAGGA
AAGATCAAAAGTGGATGATCTGAGGGGGACCCCGATGTCAGTAGGAACCTTGGAAGAGATTATTGATGACA
ATCATGCCATCGTGTCTACATCTGTGGGCTCAGAACACTACGTCAGCATTCTTTCATTTGTAGACAAGGAT
CTGCTGGAACCTGGCTGCTCGGTCCTGCTCAACCACAAGGTGCATGCCGTGATAGGGGTGCTGATGGATGA
CACGGATCCCCTGGTCACAGTGATGAAGGTAGAAAAGGCCCCCCAGGAGACCTATGCAGATATTGGGGGGT
TGGACAACCAAATTCAGGAAATTAAGGAATCTGTGGAGCTTCCTCTCACCCATCCTGAATATTATGAAGAG
ATGGGTATAAAGCCTCCTAAGGGGGTCATTCTCTATGGTCCACCTGGCACAGGTAAAACCTTGTTAGCCAA
AGCAGTAGCAAACCAAACCTCAGCCACTTTCTTGAGAGTGGTTGGCTCTGAACTTATTCAGAAGTACCTAG
GTGATGGGCCCAAACTCGTACGGGAATTGTTCCGAGTTGCTGAAGAACATGCACCGTCCATCGTGTTTATT
GATGAAATTGACGCCATTGGGACAAAAAGATATGACTCCAATTCTGGTGGTGAGAGAGAAATTCAGCGAAC
AATGTTGGAACTGCTGAACCAGTTGGATGGATTTGATTCTAGGGGAGATGTGAAAGTTATCATGGCCACAA
ACCGAATAGAAACTTTGGATCCAGCACTTATCAGACCAGGCCGCATTGACAGGAAGATTGAGTTCCCCCTG
CCTGATGAAAAGACGAAGAAGCGCATCTTTCAGATTCACACAAGCAGGATGACGCTGGCTGATGATGTAAC
CCTGGACGACCTGATCATGGCTAAAGATGACCTCTCTGGTGCTGACATCAAGGCAATCTGTACAGAAGCTG
GTCTGATGGCCTTAAGAGAACGTAGAATGAAAGTAACAAATGAAGACTTCAAAAAATCTAAAGAAAATGTT
CTTTATAAGAAACAGGAAGGCACCCCTGAGGGGCTGTATCTC
PTGFR NP_000950 prostaglandin F receptor (FP) full length (1-359)
Amino acid sequence
(SEQ ID NO: 17)
MSMNNSKQLVSPAAALLSNTTCQTENRLSVFFSVIFMTVGILSNSLAIAILMKAYQRFRQKSKASFLLLAS
GLVITDFFGHLINGAIAVFVYASDKEWIRFDQSNVLCSIFGICMVFSGLCPLLLGSVMAIERCIGVTKPIF
HSTKITSKHVKMMLSGVCLFAVFIALLPILGHRDYKIQASRTWCFYNTEDIKDWEDRFYLLLFSFLGLLAL
GVSLLCNAITGITLLRVKFKSQQHRQGRSHHLEMVIQLLAIMCVSCICWSPFLVTMANIGINGNHSLETCE
TTLFALRMATWNQILDPWVYILLRKAVLKNLYKLASQCCGVHVISLHIWELSSIKNSLKVAAISESPVAEK
SAST
Encoding nucleotide sequence
(SEQ ID NO: 18)
ATGTCCATGAACAATTCCAAACAGCTAGTGTCTCCTGCAGCTGCGCTTCTTTCAAACACAACCTGCCAGAC
GGAAAACCGGCTTTCCGTATTTTTTTCAGTAATCTTCATGACAGTGGGAATCTTGTCAAACAGCCTTGCCA
TCGCCATTCTCATGAAGGCATATCAGAGATTTAGACAGAAGTCCAAGGCATCGTTTCTGCTTTTGGCCAGT
GGCCTGGTAATCACTGATTTCTTTGGCCATCTCATCAATGGAGCCATAGCAGTATTTGTATATGCTTCTGA
TAAAGAATGGATCCGCTTTGACCAATCAAATGTCCTTTGCAGTATTTTTGGTATCTGCATGGTGTTTTCTG
GTCTGTGCCCACTTCTTCTAGGCAGTGTGATGGCCATTGAGCGGTGTATTGGAGTCACAAAACCAATATTT
CATTCTACGAAAATTACATCCAAACATGTGAAAATGATGTTAAGTGGTGTGTGCTTGTTTGCTGTTTTCAT
AGCTTTGCTGCCCATCCTTGGACATCGAGACTATAAAATTCAGGCGTCGAGGACCTGGTGTTTCTACAACA
CAGAAGACATCAAAGACTGGGAAGATAGATTTTATCTTCTACTTTTTTCTTTTCTGGGGCTCTTAGCCCTT
GGTGTTTCATTGTTGTGCAATGCAATCACAGGAATTACACTTTTAAGAGTTAAATTTAAAAGTCAGCAGCA
CAGACAAGGCAGATCTCATCATTTGGAAATGGTAATCCAGCTCCTGGCGATAATGTGTGTCTCCTGTATTT
GTTGGAGCCCATTTCTGGTTACAATGGCCAACATTGGAATAAATGGAAATCATTCTCTGGAAACCTGTGAA
ACAACACTTTTTGCTCTCCGAATGGCAACATGGAATCAAATCTTAGATCCTTGGGTATATATTCTTCTACG
AAAGGCTGTCCTTAAGAATCTCTATAAGCTTGCCAGTCAATGCTGTGGAGTGCATGTCATCAGCTTACATA
TTTGGGAGCTTAGTTCCATTAAAAATTCCTTAAAGGTTGCTGCTATTTCTGAGTCACCAGTTGCAGAGAAA
TCAGCAAGCACC
PTPRA NP_543030 protein tyrosine phosphatase, receptor type, A full
length (1-793)
Amino acid sequence
(SEQ ID NO: 19)
MDSWFILVLLGSGLICVSANNATTVAPSVGITRLINSSTAEPVKEEAKTSNPTSSLTSLSVAPTFSPNITL
GPTYLTTVNSSDSDNGTTRTASTNSIGITISPNGTWLPDNQFTDARTEPWEGNSSTAATTPETFPPSDETP
IIAVMVALSSLLVIVFIIIVLYMLRFKKYKQAGSHSNSFRLSNGRTEDVEPQSVPLLARSPSTNRKYPPLP
VDKLEEEINRRMADDNKLFREEFNALPACPIQATCEAASKEENKEKNRYVNILPYDHSRVHLTPVEGVPDS
DYINASFINGYQEKNKFIAAQGPKEETVNDFWRMIWEQNTATIVMVTNLKERKECKCAQYWPDQGCWTYGN
IRVSVEDVTVLVDYTVRKFCIQQVGDMTNRKPQRLITQFHFTSWPDFGVPFTPIGMLKFLKKVKACNPQYA
GAIVVHCSAGVGRTGTFVVIDAMLDMMHTERKVDVYGFVSRIRAQRCQMVQTDMQYVFIYQALLEHYLYGD
TELEVTSLETHLQKIYNKIPGTSNNGLEEEFKKLTSIKIQNDKMRTGNLPANMKKNRVLQIIPYEFNRVII
PVKRGEENTDYVNASFIDGYRQKDSYIASQGPLLHTIEDFWRMIWEWKSCSIVMLTELEERGQEKCAQYWP
SDGLVSYGDITVELKKEEECESYTVRDLLVTNTRENKSRQIRQFHFHGWPEVGIPSDGKGMISIIAAVQKQ
QQQSGNHPITVHCSAGAGRTGTFCALSTVLERVKAEGILDVFQTVKSLRLQRPHMVQTLEQYEFCYKVVQE
YIDAFSDYANFK
Encoding nucleotide sequence
(SEQ ID NO: 20)
ATGGATTCCTGGTTCATTCTTGTTCTGCTCGGCAGTGGTCTGATATGTGTCAGTGCCAACAATGCTACCAC
AGTTGCACCTTCTGTAGGAATTACAAGATTAATTAACTCATCAACGGCAGAACCAGTTAAAGAAGAGGCCA
AAACTTCAAATCCAACTTCTTCACTAACTTCTCTTTCTGTGGCACCAACATTCAGCCCAAATATAACTCTG
GGACCCACCTATTTAACCACTGTCAATTCTTCAGACTCTGACAATGGGACCACAAGAACAGCAAGCACCAA
TTCTATAGGCATTACAATTTCACCAAATGGAACGTGGCTTCCAGATAACCAGTTCACGGATGCCAGAACAG
AACCCTGGGAGGGGAATTCCAGCACCGCAGCAACCACTCCAGAAACTTTCCCTCCTTCAGATGAGACACCA
ATTATTGCGGTGATGGTGGCCCTGTCCTCTCTGCTAGTGATCGTGTTTATTATCATAGTTTTGTACATGTT
AAGGTTTAAGAAATACAAGCAAGCTGGGAGCCATTCCAATTCTTTCCGCTTATCCAACGGCCGCACTGAGG
ATGTGGAGCCCCAGAGTGTGCCACTTCTGGCCAGATCCCCAAGCACCAACAGGAAATACCCACCCCTGCCC
GTGGACAAGCTGGAAGAGGAAATTAACCGGAGAATGGCAGACGACAATAAGCTCTTCAGGGAGGAATTCAA
CGCTCTCCCTGCATGTCCTATCCAGGCCACCTGTGAGGCTGCTTCCAAGGAGGAAAACAAGGAAAAAAATC
GATATGTAAACATCTTGCCTTATGACCACTCTAGAGTCCACCTGACACCGGTTGAAGGGGTTCCAGATTCT
GATTACATCAATGCTTCATTCATCAACGGTTACCAAGAAAAGAACAAATTCATTGCTGCACAAGGACCAAA
AGAAGAAACGGTGAATGATTTCTGGCGGATGATCTGGGAACAAAACACAGCCACCATCGTCATGGTTACCA
ACCTGAAGGAGAGAAAGGAGTGCAAGTGCGCCCAGTACTGGCCAGACCAAGGCTGCTGGACCTATGGGAAT
ATTCGGGTGTCTGTAGAGGATGTGACTGTCCTGGTGGACTACACAGTACGGAAGTTCTGCATCCAGCAGGT
GGGCGACATGACCAACAGAAAGCCACAGCGCCTCATCACTCAGTTCCACTTTACCAGCTGGCCAGACTTTG
GGGTGCCTTTTACCCCGATCGGCATGCTCAAGTTCCTCAAGAAGGTGAAGGCCTGTAACCCTCAGTATGCA
GGGGCCATCGTGGTCCACTGCAGTGCAGGTGTAGGGCGTACAGGTACCTTTGTCGTCATTGATGCCATGCT
GGACATGATGCATACAGAACGGAAGGTGGACGTGTATGGCTTTGTGAGCCGGATCCGGGCACAGCGCTGCC
AGATGGTGCAAACCGATATGCAGTATGTCTTCATATACCAAGCCCTTCTGGAGCATTATCTCTATGGAGAT
ACAGAACTGGAAGTGACCTCTCTAGAAACCCACCTGCAGAAAATTTACAACAAAATCCCAGGGACCAGCAA
CAATGGATTAGAGGAGGAGTTTAAGAAGTTAACATCAATCAAAATCCAGAATGACAAGATGCGGACTGGAA
ACCTTCCAGCCAACATGAAGAAGAACCGTGTTTTACAGATCATTCCATATGAATTCAACAGAGTGATCATT
CCAGTTAAGCGGGGCGAAGAGAATACAGACTATGTGAACGCATCCTTTATTGATGGCTACCGGCAGAAGGA
CTCCTATATCGCCAGCCAGGGCCCTCTTCTCCACACAATTGAGGACTTCTGGCGAATGATCTGGGAGTGGA
AATCCTGCTCTATCGTGATGCTAACAGAACTGGAGGAGAGAGGCCAGGAGAAGTGTGCCCAGTACTGGCCA
TCTGATGGACTGGTGTCCTATGGAGATATTACAGTGGAACTGAAGAAGGAGGAGGAATGTGAGAGCTACAC
CGTCCGAGACCTCCTGGTCACCAACACCAGGGAGAATAAGAGCCGGCAGATCCGGCAGTTCCACTTCCATG
GCTGGCCTGAAGTGGGCATCCCCAGTGACGGAAAGGGCATGATCAGCATCATCGCCGCCGTGCAGAAGCAG
CAGCAGCAGTCAGGGAACCACCCCATCACCGTGCACTGCAGCGCCGGGGCAGGAAGGACGGGGACCTTCTG
TGCCCTGAGCACCGTCCTGGAGCGTGTGAAAGCAGAGGGGATTTTGGATGTCTTCCAGACTGTCAAGAGCC
TGCGGCTACAGAGGCCACACATGGTCCAGACACTGGAACAGTATGAGTTCTGCTACAAGGTGGTGCAGGAG
TATATTGATGCATTCTCAGATTATGCCAACTTCAAG
RAB7L1 NP_003920 RAB7, member RAS oncogene family-like 1 full
length (1-203)
Amino acid sequence
(SEQ ID NO: 21)
MGSRDHLFKVLVVGDAAVGKTSLVQRYSQDSFSKHYKSTVGVDFALKVLQWSDYEIVRLQLWDIAGQERFT
SMTRLYYRDASACVIMFDVTNATTFSNSQRWKQDLDSKLTLPNGEPVPCLLLANKCDLSPWAVSRDQIDRF
SKENGFTGWTETSVKENKNINEAMRVLIEKMMRNSTEDIMSLSTQGDYINLQTKSSSWSCC
Encoding nucleotide sequence
(SEQ ID NO: 22)
ATGGGCAGCCGCGACCACCTGTTCAAAGTGCTGGTGGTGGGGGACGCCGCAGTGGGCAAGACGTCGCTGGT
GCAGCGATATTCCCAGGACAGCTTCAGCAAACACTACAAGTCCACGGTGGGAGTGGATTTTGCTCTGAAGG
TTCTCCAGTGGTCTGACTACGAGATAGTGCGGCTTCAGCTGTGGGATATTGCAGGGCAGGAGCGCTTCACC
TCTATGACACGATTGTATTATCGGGATGCCTCTGCCTGTGTTATTATGTTTGACGTTACCAATGCCACTAC
CTTCAGCAACAGCCAGAGGTGGAAACAGGACCTAGACAGCAAGCTCACACTACCCAATGGAGAGCCGGTGC
CCTGCCTGCTCTTGGCCAACAAGTGTGATCTGTCCCCTTGGGCAGTGAGCCGGGACCAGATTGACCGGTTC
AGTAAAGAGAACGGTTTCACAGGTTGGACAGAAACATCAGTCAAGGAGAACAAAAATATTAATGAGGCTAT
GAGAGTCCTCATTGAAAAGATGATGAGAAATTCCACAGAAGATATCATGTCTTTGTCCACCCAAGGGGACT
ACATCAATCTACAAACCAAGTCCTCCAGCTGGTCCTGCTGC
SCYL3 NP_065156 SCY1-like 3 (S. cerevisiae) full length (1-688), G543A
Amino acid sequence
(SEQ ID NO: 23)
MGSENSALKSYTLREPPFTLPSGLAVYPAVLQDGKFASVFVYKRENEDKVNKAAKHLKTLRHPCLLRFLSC
TVEADGIHLVTERVQPLEVALETLSSAEVCAGIYDILLALIFLHDRGHLTHNNVCLSSVFVSEDGHWKLGG
METVCKVSQATPEFLRSIQSIRDPASIPPEEMSPEFTTLPECHGHARDAFSFGTLVESLLTILNEQVSADV
LSSFQQTLHSTLLNPIPKCRPALCTLLSHDFFRNDFLEVVNFLKSLTLKSEEEKTEFFKFLLDRVSCLSEE
LIASRLVPLLLNQLVFAEPVAVKSFLPYLLGPKKDHAQGETPCLLSPALFQSRVIPVLLQLFEVHEEHVRM
VLLSHIEAYVEHFTQEQLKKVILPQVLLGLRDTSDSIVAITLHSLAVLVSLLGPEVVVGGERTKIFKRTAP
SFTKNTDLSLEGDPFSQPIKFPINGLSDVKNTSEDSENFPSSSKKSEEWPDWSEPEEPENQTVNIQIWPRE
PCDDVKSQCTTLDVEESSWDDCEPSSLDTKVNPGGGITATKPVTSAEQKPIPALLSLTEESMPWKSSLPQK
ISLVQRGDDADQIEPPKVSSQERPLKVPSELGLGEEFTIQVKKKPVKDPEMDWFADMIPEIKPSAAFLILP
ELRTEMVPKKDDVSPVMQFSSKFAAAEITEGEAEGWEEEGELNWEDNNW
Encoding nucleotide sequence
(SEQ ID NO: 24)
ATGGGATCAGAGAACAGTGCTTTAAAGAGCTATACACTGAGAGAACCACCATTTACCTTACCCTCTGGACT
TGCTGTTTATCCCGCTGTACTGCAAGATGGCAAATTTGCTTCAGTTTTTGTGTATAAGAGAGAAAATGAAG
ACAAGGTTAATAAAGCTGCCAAGCATTTGAAGACACTTCGTCACCCTTGCTTGCTAAGATTTTTATCTTGT
ACTGTGGAAGCGGATGGCATTCATCTTGTCACTGAGCGAGTACAGCCCCTGGAAGTGGCTTTGGAAACATT
GTCTTCTGCAGAGGTCTGTGCTGGGATCTATGACATATTGCTGGCTCTTATCTTCCTTCATGACAGAGGAC
ACCTAACACACAATAATGTCTGTTTATCATCTGTGTTTGTGAGTGAAGATGGACACTGGAAGCTAGGAGGA
ATGGAAACTGTTTGTAAAGTTTCTCAGGCCACACCAGAGTTTCTGAGGAGTATTCAGTCAATAAGAGACCC
AGCATCTATCCCTCCTGAAGAGATGTCTCCAGAATTCACAACTCTCCCAGAGTGTCATGGACATGCCCGGG
ATGCCTTTTCATTTGGAACATTGGTGGAAAGTTTGCTCACAATCTTAAATGAACAGGTTTCAGCGGATGTT
CTCTCCAGCTTTCAACAGACCTTGCACTCAACTTTGCTGAATCCCATTCCAAAATGTCGGCCAGCGCTCTG
CACCTTACTATCTCATGACTTCTTCAGAAATGATTTTCTGGAAGTTGTGAATTTCTTGAAAAGTTTAACAT
TGAAGAGTGAAGAGGAGAAAACGGAATTCTTTAAATTTCTGCTGGACAGAGTCAGCTGCTTGTCAGAGGAA
TTGATAGCTTCAAGGTTGGTGCCTCTTCTGCTTAATCAGTTGGTGTTTGCAGAGCCAGTGGCTGTTAAGAG
TTTTCTTCCTTATCTGCTTGGCCCCAAAAAAGATCATGCGCAGGGAGAAACTCCTTGCTTGCTCTCACCAG
CCCTGTTCCAGTCACGGGTGATCCCCGTGCTTCTCCAGTTGTTTGAAGTTCATGAAGAGCATGTGCGGATG
GTGCTGCTGTCTCACATCGAGGCCTACGTGGAGCACTTCACTCAGGAGCAGCTGAAGAAAGTCATCTTGCC
ACAGGTTTTGCTGGGCCTGCGTGATACTAGTGATTCCATTGTGGCAATTACTCTGCATAGCCTAGCAGTGC
TGGTCTCTCTGCTTGGACCAGAGGTGGTTGTGGGAGGAGAACGAACCAAGATCTTCAAACGCACTGCCCCA
AGTTTTACTAAAAATACTGACCTTTCTCTAGAAGGTGATCCATTTTCTCAGCCTATTAAATTTCCCATAAA
CGGACTCTCAGATGTAAAAAATACTTCGGAGGACAGTGAAAACTTCCCATCAAGTTCTAAAAAGTCTGAGG
AGTGGCCTGACTGGAGTGAACCTGAGGAGCCTGAAAATCAAACTGTCAACATACAGATTTGGCCTAGAGAA
CCTTGTGATGATGTCAAGTCCCAGTGCACTACCTTGGATGTGGAAGAGTCATCTTGGGATGACTGCGAGCC
CAGCAGCTTAGATACTAAAGTAAACCCAGGAGGTGGAATCACTGCTACAAAACCTGTTACCTCAGCGGAGC
AGAAGCCTATTCCTGCTTTGCTTTCACTCACTGAAGAGTCTATGCCTTGGAAATCAAGCTTACCCCAAAAG
ATTAGCCTTGTACAAAGGGGGGATGACGCAGACCAAATCGAGCCGCCAAAAGTGTCATCACAAGAAAGGCC
CCTTAAGGTTCCATCAGAACTTGGTTTAGGAGAGGAATTCACCATTCAAGTAAAAAAGAAGCCAGTAAAAG
ATCCTGAGATGGATTGGTTTGCTGATATGATCCCAGAAATTAAGCCTTCTGCTGCTTTTCTTATATTACCT
GAACTGAGGACAGAAATGGTCCCAAAAAAGGATGATGTCTCCCCAGTGATGCAGTTTTCCTCAAAATTTGC
TGCAGCAGAAATTACTGAGGGAGAGGCTGAAGGCTGGGAAGAAGAAGGGGAGCTGAACTGGGAAGATAATA
ACTGG

The claims are not meant to be limited to the materials and methods, embodiments, and examples described herein.

1. A method for detecting ovarian cancer onset, comprising the steps of:
contacting a fluid from a patient that contains immunoglobulins with a substrate having one or more biomarkers selected from the group consisting of ACSBG1, AFP, CSNK1A1L, DHFR, MBNL1, TP53, PRL, PSMC1, PTGFR, PTPRA, RAB7L1, and SCYL3; and
detecting whether one or more of said biomarkers is bound by said immunoglobulins.

2. The method of claim 1, wherein said substrate one or more biomarkers comprises a Nucleic Acid Protein Programmable Array (NAPPA).

3. A diagnostic test kit for ovarian cancer, comprising a substrate including one or more biomarkers selected from the group consisting of ACSBG1, AFP, CSNK1A1L, DHFR, MBNL1, P53, PRL, PSMC1, PTGFR, PTPRA, RAB7L1, and SCYL3.

4. A diagnostic test kit for ovarian cancer, comprising a substrate including biomarkers ACSBG1, AFP, CSNK1A1L, DHFR, MBNL1, P53, PRL, PSMC1, PTGFR, PTPRA, RAB7L1, and SCYL3.
